(19) United States
(12) Patent Application Publication
(10) Pub. No.: US 2003/0199082 A1
(43) Pub. Date: Oct. 23, 2003

(54) Thermally-conductive biological assay trays

(76) Inventors: James Miller, Marietta, GA (US); E. Mikhail Sagal, Wakefield, RI (US); Kevin McCullough, North Kingstown, RI (US)

Correspondence Address: BARLOW, JOSEPHS & HOLMES, LTD., 101 DYER STREET, 5TH FLOOR, PROVIDENCE, RI, 02903, US

(21) Appl. No.: 10/409,470

(22) Filed: Apr. 8, 2003

Related U.S. Application Data
(60) Provisional application No. 60/373,014, filed on Apr. 15, 2002.

(51) Int. Cl. C12M 1/34

(52) U.S. Cl. 435/288.5; 422/102

(57) ABSTRACT

A thermally-conductive biological assay tray is provided. The trays are made from a polymer composition comprising a base polymer matrix and a thermally-conductive material. The trays can be used for fluorescent immunoassays. The fluorescence level of the polymer composition is sufficiently low such that it does not interfere with the fluorescent immunoassay process. The invention also includes methods for making the bioassay trays.

### CROSS-REFERENCE TO RELATED APPLICATIONS

The application claims the benefit of U.S. Provisional Application No. 60/373,014 having a filing date of Apr. 15, 2002.

### BACKGROUND OF THE INVENTION

The present invention relates generally to biological assay trays. Particularly, the present invention relates to thermally-conductive, biological assay trays and methods for making such trays. The trays are made from polymer compositions comprising a base polymer matrix and a thermally-conductive material.

Biochemical research and medical laboratories use biological assay trays for various purposes including analyzing and testing genetic materials, cells, tissue cultures, immunological complexes, and the like. In general, biological assays are used to detect the presence or concentration of a substance (for example, a protein) in a sample material.

These assays are commonly performed in receptacle trays containing multiple wells arranged in rows and columns. The tray typically contains 20, 24, 48, or 96 wells with each well holding fluids in microliter quantities. The wells can have various shapes. The upper portion of the well is round usually, although square-shaped wells are also known. The bottom portion of the well can be flat, round, V-shaped, or U-shaped. Biological assays involve a sequence of steps depending on the specific type of assaying technique being performed. In general, these techniques involve placing a fluid sample that will be analyzed into the wells in the tray, adding various liquid reagents, incubating and cooling the samples, washing the reacted samples multiple times, and other steps. The addition of the liquid reagents and washings are usually conducted using manual or automated pipettes.

Immunoassays are frequently used to analyze biological materials. Many immunoassay procedures involve forming an antigen-antibody complex. Antigens are agents that stimulate the formation of a corresponding antibody. Immunoassay procedures can be used to determine the presence of antigens in bodily fluids such as whole blood, serum, plasma, and urine. In general, antibodies refer to any of the body immunoglobulins that are produced in response to specific antigens. Specific antibodies react with specific antigens to form a binding antigen-antibody complex. These binding reactions often cause precipitation or agglutination which can be visible to the naked eye in the sample. However, in many instances, special instruments must be used to analyze the presence of such antigen-antibody complexes.

In many immunoassays, one of the components of the complex (for example, antigen or antibody) is immobilized on a solid support surface located inside the wells of the assay tray. This results in the entire complex being immobilized on the solid support surface. The immobilized, solid-phase complexes in the tray wells can be washed, incubated, isolated, and treated with liquid reagents. These assays are commonly referred to as immunosorbent or solid phase assays. Conventional solid phase assays include, for example, enzyme immunoassays (EIAs), radio immunoassays (RIAs), and fluorescent immunoassays (FIAs) in which the immunosorbent material is some type of bead, disc, or other solid support material.

As discussed above, immunoassays and other biological assays involve heating and cooling the tray several times so that the contents of the tray are incubated and cooled to the proper temperatures. The time required to heat and cool the tray is a factor in determining how many analytical measurements are made in a given period. The heating and cooling time periods impact the costs and efficiencies of the analytical tests. With metal assay trays, the heating and cooling steps are performed quickly. However, most metals interfere with the reactants in the tray wells or the detection methods used; therefore, metal assay trays are not commonly used. Even if a metal tray (for example, a stainless steel or titanium tray) does not interfere with the reactants, it is costly to manufacture such trays. Further, many laboratories want to dispose of biological assay trays after a single use. Fabricating metal assay trays for single applications is very costly.

Thus, biochemical research and medical laboratories typically use plastic biological assay trays. These assay trays are made from biologically inert materials and relatively inexpensive to manufacture. For example, the tray can be made from polymers such as polystyrene, polyethylene, polypropylene, acrylates, methacrylates, acrylics, polyacrylamides, and vinyl polymers such as vinyl chloride and polyvinyl fluoride.

Many such plastic assay trays are made using known injection-molding processes, and the trays can have various configurations.

For example, Astle, U.S. Pat. No. 5,225,164 discloses a microplate tray with open-top wells having a rectilinear shape for analyzing liquid reagents and other sample materials. The wells may contain baffles to promote mixing and increase the rate of oxygen transfer to the liquid in the wells. The Patent discloses that the elements of the tray can be constructed from molded polystyrene.

Peters, U.S. Pat. No. 4,299,920 discloses a receptacle for cell cultures or biological tests comprising a base plate, and a wall member joined in a detachable and liquid-tight manner to the base plate. The Patent discloses that the base plates are flexible and can be made of polystyrene, polycarbonate, fluorinated polymerized hydrocarbons, or glass. The Patent further discloses that the wall section can be made from an elastomeric synthetic material such as polyvinylchloride, polyurethane elastomers, polyvinylidene chloride, methyl rubber, chlorinated rubber, or fluorocarbon elastomers.

Studer, Jr., U.S. Pat. No. 4,090,920 discloses a biological culture test plate having a plurality of test wells or chambers. The test plate is a disposable, transparent structure made from a molded plastic. The Patent discloses that the molded plate can be made from methyl methacrylate, vinyl resin, or any biologically inert polymer.

Katoh et al., U.S. Pat. No. 6,319,475 discloses a container for holding sample materials in which the container is subjected to a thermal heating and cooling process. The container can be used in the medical, chemical, and biotechnology fields. The container comprises three layers including a layer made of a composition containing a resin and inorganic filler selected from the group consisting of ceramics, metals, and carbons.

However, conventional plastic assay trays have some drawbacks. Particularly, conventional plastic assay trays generally have poor thermal-conductive properties. The thermal heating and cooling efficiency of assays using such known plastic trays can be low. In fact, many plastic trays are designed for the purpose of having good thermal-insulation properties. However, the time period for heating and cooling such plastic trays can be relatively long, and this increases the costs of the assaying process. In addition, plastic trays having poor thermal-conductive properties may not transfer heat uniformly to the wells in the tray. This non-uniform heating of the tray may cause temperature gradients to occur between the wells and impact analysis of the contents in the wells.

In view of the foregoing disadvantages with conventional biological assay trays, there is a need for an improved assay tray having good thermal-conductive properties. It would be desirable to have an assay tray which could be heated and cooled rapidly to improve the efficiency of the assays. The present invention provides such biological assay trays and methods for making such trays.

### SUMMARY OF THE INVENTION

This invention relates to relates to thermally-conductive biological assay trays and methods for making such trays.

In general, the thermally-conductive polymer composition comprises: a) 20% to 80% by weight of a polymer matrix, and b) 20% to 80% by weight of a non-metallic, thermally-conductive material. The polymer matrix can be a thermoplastic or thermosetting polymer. For example, polyphenylene sulfide can be used to form the polymer matrix. The non-metallic, thermally-conductive material is preferably selected from ceramics, oxides, and carbon materials. For example, the thermally-conductive material can be boron nitride, silicon nitride, alumina, silicon oxide, magnesium oxide, or carbon graphite.

A molten polymer composition is provided, and the composition is injected into a mold. The composition is then removed from the mold to form a net-shape molded, thermally-conductive, biological assay tray.

Preferably, the biological assay tray has a thermal-conductivity of greater than 3 W/m°K., and more preferably greater than 22 W/m°K.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description taken in connection with the accompanying drawing in which:

FIG. 1 is a perspective view of a biological assay tray made from a thermally-conductive polymer in accordance with the present invention; and

FIG. 2 is a perspective view of a single test well disposed within the assay tray of FIG. 1.

### DETAILED DESCRIPTION OF THE PREFERRED EMBODIMENTS

The present invention relates to thermally-conductive, biological assay trays and methods for making such trays. The trays are made using polymer compositions having high thermal-conductivity. The polymer composition comprises a polymer matrix and thermally-conductive material dispersed therein.

In one standard fluorescent “sandwich” immunoassay technique, the bioassay tray well contains an immunosorbent support surface (for example an agarose-coated glass disc or beads). An unlabelled antibody that will react with the antigens to be analyzed is immobilized on the porous glass disc. A fluid containing the antigens is fed through the disc so that the antigen molecules react and bind to the immobilized antibodies. Next, a solution containing antibody molecules that have been labeled with a detectable fluorescent label (for example, a fluorescein molecule) is fed through the porous glass disc. The labeled antibody molecules bind to the antigen molecules to form a sandwich-layered structure on the disc. The layered structure comprises unlabeled antibodies, antigen, and labeled antibodies. A spectrofluorometer is used to measure the presence and concentration of the labeled antibody molecules.

In another known fluorescent immunoassay procedure, antigens of the same immunological type of antigen in the fluid to be analyzed, are adsorbed on the support disc. The support disc containing the adsorbed antigens is immersed in a solution containing labeled antibodies and the antigens to be analyzed. The labeled antibodies react and bind rapidly to the antigens in solution so that this reaction goes to completion. Excess labeled antibodies which are not bound to the antigens in the solution will react with the antigens immobilized on the support surface. Next, the support surface can be washed in a buffer solution. Then, the support surface can be analyzed for the presence of labeled antibody-antigen complexes using a fluorometer or other appropriate instrument.

In such fluorescent immunoassay techniques, it is important that the base polymer comprising the tray have a relatively low level of fluorescence so that the background fluorescence can be kept to a minimum and not interfere with the test readings. The background fluorescence can disguise actual fluorescence levels making it difficult to obtain accurate readings. In other words, the fluorescence level of the base polymer is sufficiently low such that it does not interfere with the fluorescent immunoassay process. A thermoplastic polymer selected from the group consisting of polycarbonates, polyethylene, polypropylene, acrylics, vinyls, fluorocarbons, polyamides, polyesters, polyphenylene sulfide, and liquid crystal polymers such as thermoplastic aromatic polyesters can be used to form the matrix. Liquid crystal polymers having a sufficiently low fluorescence so as not to interfere with the reading of the fluorescence levels of the labeled antibody-antigen complexes is particularly preferred. Alternatively, thermosetting polymers such as elastomers, epoxies, polyimides, and acrylonitriles can be used. Suitable elastomers include, for example, styrene-butadiene copolymer, polychloroprene, nitrile rubber, butyl rubber, polysulfide rubber, ethylene-propylene terpolymers, polysiloxanes (silicones), and polyurethanes. Generally, the polymer matrix comprises about 20 to about 80% by weight of the total composition and more particularly about 40 to about 80% by weight of the composition.

In the present invention, non-metallic, thermally-conductive materials are added and dispersed within the polymer matrix. These materials impart thermal conductivity to the non-conductive polymeric matrix. It is important that nonmetallic materials be used, because metals metal contaminates can react and bind with the reactants in the tray wells causing analytical problems. Further, the thermally-conductive materials should have low fluorescence so that background fluorescence levels are kept to a minimum for the reasons discussed above.

Suitable non-metallic, thermally-conductive materials include, metal oxides such as alumina, magnesium oxide, zinc oxide, and titanium oxide; ceramics such as silicon nitride, aluminum nitride, boron nitride, boron carbide, and carbon materials such as carbon black or graphite. Mixtures of such fillers are also suitable. Generally, the thermally-conductive fillers comprise about 20 to about 80% by weight of the total composition and more particularly about 30 to about 60% by weight of the composition.

The thermally conductive material can be in the form of particles, granular powder, whiskers, fibers, or any other suitable form. The particles or granules can have a variety of structures and a broad particle size distribution. For example, the particles or granules can have flake, plate, rice, strand, hexagonal, or spherical-like shapes with a particle size in the range of 0.5 to 300 microns. Preferably, the particle size is small (e.g., <1 micron), because such particles tend not to reflect the beam of light from the fluorometer or other instrument reading the samples as discussed in further detail below. In some instances, the thermally conductive material can have a relatively high aspect (length to thickness) ratio of about 10:1 or greater. For example, PITCH-based carbon fiber having an aspect ratio of about 50:1 can be used. Alternatively, the thermally conductive material can have a relatively low aspect ratio of about 5:1 or less. For example, boron nitride grains having an aspect ratio of about 4:1 can be used. Both low aspect and high aspect ratio materials can be added to the polymer matrix as described in McCullough, U.S. Pat. No. 6,048,919, the disclosure of which is hereby incorporated by reference. Particularly, the compositions of this invention can contain about 25 to about 60% by weight of a thermally conductive material having a high aspect ratio of about 10:1 or greater, and about 10 to about 25% by weight of a thermally conductive material having a low aspect ratio of about 5:1 or less.

An optional reinforcing material can be added to the polymer matrix. The reinforcing material can be glass, inorganic minerals, or other suitable material. The reinforcing material strengthens the polymer matrix. The reinforcing material, if added, constitutes about 3% to about 25% by weight of the composition.

The thermally-conductive material and optional reinforcing material are intimately mixed with the non-conductive polymer matrix to form the polymer composition. If desired, the mixture may contain additives such as, for example, flame retardants, antioxidants, plasticizers, dispersing aids, and mold-releasing agents. Preferably, such additives are biologically inert. The mixture can be prepared using techniques known in the art.

Also, as discussed above, in some types of assays such as fluoroimmunoassays and enzyme immunoassays, the reading step of the assay involves passing a beam of light through the wells in the tray and “reading” the contents of the wells. The polymer compositions of the present invention used to make the bio-assay trays tend not to interfere with the incident light beams, particularly the polymer compositions tend not to reflect the light beams. Thus, more accurate readings and measurements can be made. In some instances, the polymer composition can be colored black using carbon black so that the composition acts more effectively as an ultraviolet (UV) light absorber and reduces reflection of the light beam.

Preferably, the polymer compositions have a thermal conductivity of greater than 3 W/m°K and more preferably greater than 22 W/m°K. These good heat-conduction properties allow the assay tray to be efficiently heated and cooled. Further, since the polymer composition used to make the bioassay tray has good thermal-conductivity properties, heat can be uniformly transferred to all of the wells in the tray. Thus, there is less likely to be significant temperature differences between the wells, and more accurate readings can be obtained.

The resulting polymer composition can be shaped into the bioassay tray using any suitable molding process such as melt-extrusion, casting, or injectionmolding.

In general, injection-molding involves the steps of: a) feeding the composition into the heating chamber of a molding machine and heating the composition to form a molten composition (liquid plastic); b) injecting the molten composition into a mold cavity; c) maintaining the composition in the mold under high pressure until it cools; and d) removing the molded article.

The molding process produces a “net-shape molded” bioassay tray. The final shape of the bioassay tray is determined by the shape of the mold cavity. No further processing, die-cutting, machining, or other tooling is required to produce the final shape of the bioassay tray.

It should be recognized that the bioassay trays of the present invention have a single-layered construction. The thermally conductive polymer composition is molded into the shape of the tray assembly comprising a flat platform with test wells disposed therein. The tray assembly (platform and wells) is an integrated unitary structure made from a polymer composition as described above. The tray assembly does not comprise an interior layer which is made from a first polymer composition having one degree of thermal conductivity, and an exterior layer made from a second polymer composition having a different degree of thermal conductivity.

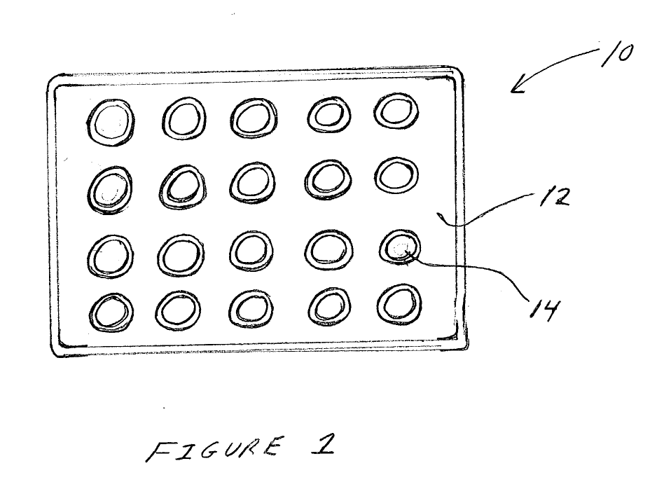

The bioassay trays can have various shapes and structures depending on the type of bioassay tray desired. For example, a thermally-conductive bioassay tray having the design shown in FIG. 1 can be made in accordance with this invention. In FIG. 1, the biological assay tray is generally indicated at 10. The tray comprises a flat platform 12 containing multiple test wells (recessed portions) 14 disposed therein. The test wells are arranged in rows and columns.

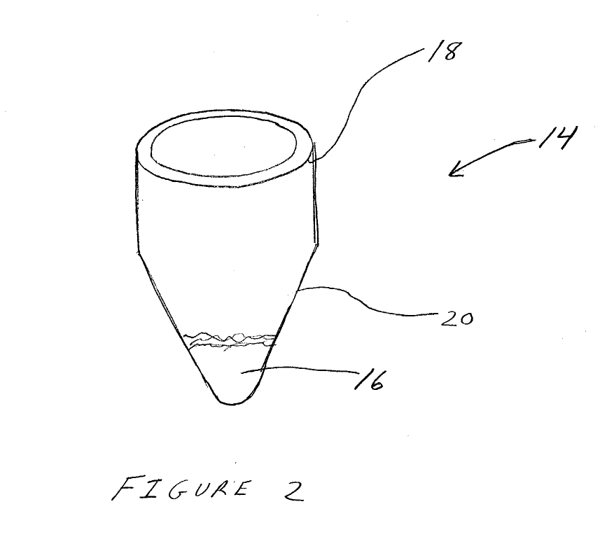

In FIG. 2, a single test well 14 containing sample fluid 16 is shown. The test well 14 has a rounded upper portion 18 and a V-shaped lower portion 20. It is understood that the test wells 14 can have structures other than the designs shown in FIG. 2. There is a wide variety of suitable structures for the test wells 14. For example, the upper portion of the well can have a square shape and the lower portion of the well can have a round, flat, or U-shaped structure.

The bioassay trays of the present invention have good thermal conductive properties. Preferably, the tray has a thermal-conductivity of greater than 3 W/m°K and more preferably greater than 22 W/m°K. The heating and cooling steps of a wide variety of immunoassays can be performed efficiently using the assay trays of the present invention.

It is appreciated by those skilled in the art that various changes and modifications can be made to the illustrated embodiments without departing from the spirit of the invention. All such modifications and changes are intended to be covered by the appended claims.

What is claimed is:

1. A thermally-conductive, biological assay tray comprising a platform having multiple test wells disposed therein, said platform comprising a polymer composition, said composition comprising: i) about 20% to about 80% by weight of a polymer matrix, and ii) about 20% to about 80% by weight of a non-metallic, thermally-conductive material.

2. The assay tray of claim 1, wherein the tray has a thermal conductivity of greater than 3 W/m°K.

3. The assay tray of claim 1, wherein the polymer matrix comprises a thermoplastic polymer.

4. The assay tray of claim 3, wherein the thermoplastic polymer is selected from the group consisting of polycarbonates, polyethylene, polypropylene, acrylics, vinyls, fluorocarbons, polyamides, polyesters, polyphenylene sulfide, and liquid crystal polymers.

5. The assay tray of claim 1, wherein the polymer matrix comprises a thermosetting polymer.

6. The assay tray of claim 1, wherein the thermally-conductive material is selected from the group consisting of ceramics, metal oxides, and carbon materials.

7. The assay tray of claim 6, wherein the thermally-conductive material is selected from the group consisting of silicon nitride, boron nitride, alumina, magnesium oxide, and carbon graphite.

8. The assay tray of claim 1, wherein the polymer composition further comprises: (iii) a reinforcing material.

9. The method of claim 8, wherein the reinforcing material is glass.

10. A method of making a net-shape molded, thermally-conductive biological assay tray, comprising the steps of:
a) providing a molten composition comprising: i) about 20% to about 80% by weight of a polymer matrix, and ii) about 20% to about 80% by weight of a non-metallic, thermally-conductive material;
b) injecting the molten composition into a mold;
c) removing the composition from the mold to form a net-shape molded, thermally-conductive biological assay tray comprising a platform having multiple test wells disposed therein.

11. The method of claim 10, wherein the assay tray has a thermal conductivity of greater than 3 W/m°K.

12. The method of claim 10, wherein the polymer matrix comprises a thermoplastic polymer.

13. The method of claim 11, wherein the thermoplastic polymer is selected from the group consisting of polycarbonates, polyethylene, polypropylene, acrylics, vinyls, fluorocarbons, polyamides, polyesters, polyphenylene sulfide, and liquid crystal polymers.

14. The method of claim 10, wherein the polymer matrix comprises a thermosetting polymer.

15. The method of claim 10, wherein the thermally-conductive material is selected from the group consisting of ceramics, metal oxides, and carbon materials.

16. The method of claim 15, wherein the thermally-conductive material is selected from the group consisting of silicon nitride, boron nitride, alumina, magnesium oxide, and carbon graphite.

17. The method of claim 10, wherein the composition further comprises reinforcing material.
